(19) United States
(12) Patent Application Publication
(10) Pub. No.: US 2025/0011817 A1
(43) Pub. Date: Jan. 9, 2025

(54) METHOD FOR REDUCING FERMENTATION BROTH VISCOSITY

(71) Applicants: PURAC BIOCHEM B.V., GORINCHEM, (NL); CORBION BIOTECH, INC., South San Francisco, CA (US)

(72) Inventors: Lazar DRASKOVIC, Gorinchem (CA); Thomas WILLIAMS, South San Francisco, CA (US); John PIECHOCKI, South San Francisco, CA (US); Jorge GALAZZO, South San Francisco, CA (US)

(21) Appl. No.: 18/887,813

(22) Filed: Sep. 17, 2024

Related U.S. Application Data
(63) Continuation of application No. PCT/EP2023/056882, filed on Mar. 17, 2023.

(30) Foreign Application Priority Data
Mar. 18, 2022 (EP) 22163035.3

(51) Int. Cl. C12P 1/00; C12N 1/02; C12N 1/06; C12N 1/12

(52) U.S. Cl. CPC C12P 1/00; C12N 1/02; C12N 1/06; C12N 1/12

(57) ABSTRACT

The invention pertains to a method for reducing the viscosity of a fermentation broth, wherein the method comprises the step of mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated on the total weight of the fermentation broth, wherein the total lipid content of the dry matter content of the fermentation broth is at least 40 wt. %. The invention also pertains to a fermentation broth with a low viscosity, methods for processing the fermentation broth with a low viscosity, a dried biomass, and a use of the fermentation broth with a low viscosity in the preparation of a dried biomass, an extracted microbial oil, a feed additive, or of an animal feed.

### RELATED PATENT APPLICATIONS

The present application is a continuation application of PCT/EP2023/056882 filed Mar. 17, 2023, which claims priority to EP 22163035.3 filed Mar. 18, 2022, the entire contents of both which are incorporated herein by reference.

### TECHNICAL FIELD OF THE INVENTION

The present invention relates to a method for reducing the viscosity of a fermentation broth, a fermentation broth having a low viscosity, a method for processing the fermentation broth with a low viscosity, and a use of the fermentation broth with a low viscosity in the preparation of dried biomass, an extracted microbial oil, a feed additive, or an animal feed.

### BACKGROUND AND SUMMARY OF THE INVENTION

When a fermentation process ends, the fermentation broth obtained by the fermentation process generally needs to be processed in order to recover a desired fermentation product(s) from the fermentation broth, such as biomass or a microbial oil. Some processing steps require that the fermentation broth has specific properties, such as a low viscosity.

For example, CN 101168501 describes a process for extracting and purifying docosahexaenoic acid (DHA)-rich fatty acids from Crypthecodinium cohnii. It is said that the viscosity of the fermentation broth was too high for certain downstream processing steps, in particular centrifugation to remove water. It is explained that subjecting the viscous fermentation broth to these downstream processing steps would be too energy-consuming and would lead to oxidation of the fermentation product (DHA). To circumvent the problems associated with processing fermentation broths having a high viscosity, an alternative process is presented in which a flocculant is added to the fermentation broth in order to flocculate and precipitate the cells, and water is subsequently removed. Aluminum sulfate and ferric chloride are examples of flocculating agents used.

Other approaches to changing the viscosity of a fermentation broth are described in WO 2005/063999. This document describes a process for producing an oil that involves deaerating an aqueous liquid comprising cells (e.g., a fermentation broth) from which the oil is (later) obtained. The aqueous liquid can be deaerated by changing its viscosity. It is said that viscosity can be changed by dilution with water (or other liquids) or by means of a temperature change.

There is a need in the art for a method for reducing the viscosity of a biomass-containing fermentation broth that does not require flocculation of the biomass, dilution of the fermentation broth or heating of the fermentation broth, as these approaches are not always suitable for industrial processes. For example, dilution of the fermentation broth requires that the equipment for downstream processes can accommodate large volumes of fermentation broth. This, in turn, requires investments into such equipment.

It is an object of the invention to provide a method for reducing the viscosity of a fermentation broth that accommodates this need, is easy to operate, and is economical. The present invention provides such a method.

In an aspect, the invention relates to a method for reducing the viscosity of a fermentation broth, wherein the method comprises the step of
mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated on the total weight of the fermentation broth, wherein the total lipid content of the dry matter content of the fermentation broth is at least 40 wt. %.

It has been found that the viscosity of a biomass-containing fermentation broth containing few to no lysed microbial cells (i.e., a fermentation broth wherein most of the microbial cells are whole cells and, thus, the amount of free microbial oil is low) can surprisingly be reduced by addition of one or more specific organic acids or their salts. This could not have been expected, as the addition of other acids and their salts was found increase the viscosity of fermentation broths (as shown in the Examples). The specific organics acids and their salts that lead to a viscosity reduction can conveniently be added at the end of a fermentation process.

In another aspect, the invention relates to a fermentation broth comprising microbial cells, at least one compound selected from the group consisting of ascorbic acid, citric acid, lactic acid, formic acid, amino acids, and soluble salts of said acids, and 5.0 wt. % or less of free microbial oil (calculated on the total weight of the fermentation broth), wherein the total lipid content of the dry matter content of the fermentation broth is at least 40 wt. %, optionally wherein the fermentation broth has a viscosity of 1.0 Pa·s or less, measured at a shear rate of 9.8-10.0 s 1, 25° C., and according to a shear rate logarithmic ramp up and down isothermal sweep (shear rate range of 0.1-100 s⁻¹) using a Malvern Kinexus Pro+ rheometer with a cup and bob system.

As a result of the reduced viscosity of the fermentation broth (obtained) according to the invention, downstream processing of the fermentation broth is improved. For example, as will be discussed in more detail below, conventional evaporation methods can be used to dewater the broth, resulting in a concentrated broth with higher dry matter content. Dewatering by evaporation significantly reduces the volume of liquid to be handled during subsequent processing steps, which provides certain advantages. For example, the reduced volume of liquid after evaporation step allows the use of smaller centrifuges to separate the oil and aqueous phases during subsequent oil extraction steps. Alternatively, it increases the throughput of drying during subsequent dry biomass generation steps. Overall, the reduced viscosity of the fermentation broth due to the addition of at least one compound provided herein allows for a more efficient and more versatile method for the recovery of desired fermentation products, such as DHA.

Accordingly, the fermentation broth obtained by the method of the claimed invention, and the fermentation broth of the claimed invention, can be subjected to further processing in a variety of manners. Therefore, in yet another aspect, the invention relates to a method for processing a fermentation broth obtained by the method for reducing the viscosity of a fermentation broth according to the invention or a fermentation broth according to the invention, wherein the method comprises one of the following process sequences:
(a) concentrating the fermentation broth (e.g. by evaporation (of water), filtration, or centrifugation) to obtain a concentrated fermentation broth;
(b) concentrating the fermentation broth (e.g. by evaporation (of water), filtration, or centrifugation) to obtain a concentrated fermentation broth, and, thereafter, drying the concentrated fermentation broth to obtained a dried biomass;
(c) concentrating the fermentation broth (e.g. by evaporation (of water), filtration, or centrifugation) to obtain a concentrated fermentation broth, drying the concentrated fermentation broth to obtained a dried biomass, and lysing the dried biomass (optionally in the presence of oil) to obtain a dispersion comprising lysed microbial cells in oil;
(d) concentrating the fermentation broth (e.g. by evaporation (of water), filtration, or centrifugation) to obtain a concentrated fermentation broth, lysing microbial cells in the concentrated fermentation broth to obtain a concentrated fermentation broth comprising lysed microbial cells, and separating microbial oil from the concentrated fermentation broth comprising lysed microbial cells in one or more steps, optionally without performing a demulsification step (e.g., adding an additional demulsification agent) before separating the microbial oil;
(e) drying the fermentation broth (without first concentrating the fermentation broth) to obtain a dried biomass;
(f) drying the fermentation broth (without first concentrating the fermentation broth) to obtain a dried biomass and, thereafter, lysing the dried biomass (optionally in the presence of oil) to produce a dispersion comprising lysed microbial cells in oil;
(g) lysing microbial cells in the fermentation broth to obtain a fermentation broth comprising lysed cells;
(h) lysing microbial cells in the fermentation broth to obtain a fermentation broth comprising lysed cells and separating microbial oil from the fermentation broth comprising lysed cells in one or more steps, optionally wherein a demulsification step is performed prior to separating microbial oil from the fermentation broth comprising lysed cells; or
(i) lysing microbial cells in the fermentation broth to obtain a fermentation broth comprising lysed cells, concentrating the fermentation broth comprising lysed cells (e.g., by evaporation (of water) or centrifugation), and separating microbial oil from the fermentation broth comprising lysed cells in one or more steps, optionally wherein a demulsification step is performed prior to separating microbial oil from the fermentation broth comprising lysed cells.

In still another aspect, the invention relates to a dried biomass comprising microbial cells, 5 wt. % or less of water (calculated on the total weight of the dried biomass), and a total amount of from 0.1 to 20 wt. % of at least one compound selected from the group consisting of ascorbic acid, citric acid, lactic acid, formic acid, amino acids, and soluble salts of said acids (calculated on the total weight of the dried biomass), wherein the total lipid content of the dry matter content of the biomass is at least 40 wt. %. The amounts of water, the at least one compound, and the microbial cells are based on the total weight of the dried biomass.

The addition of the at least one compound to the fermentation broth reduces the viscosity of the fermentation broth without negatively impacting its free microbial oil content. A fermentation broth with a lower viscosity allows an easier application of downstream processing steps, such as evaporation. After the evaporation step, the concentrated fermentation broth would require less residence time and/or heat in a dryer to produce a dried biomass. As a result, the dried biomass, after the drying process, could have improved properties, such as flowability or a higher quality microbial oil content (due to a relatively low free microbial oil content).

In a further aspect, the invention relates to a use of the fermentation broth according to the invention in the preparation of dried biomass, an extracted microbial oil, a feed additive, or an animal feed. The invention also relates to a use of the dried biomass according to the invention in the preparation of an extracted microbial oil, a feed additive, or an animal feed.

### DETAILED DESCRIPTION

The aspects of the invention will be discussed in more detail below. Specific advantages of the aspects of the invention, as well as of specific embodiments thereof, will become apparent from the further specification.

### Method for Reducing the Viscosity of a Fermentation Broth

As mentioned above, disclosed herein is a method for reducing the viscosity of a fermentation broth, wherein the method comprises the step of
mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated on the total weight of the fermentation broth, wherein the total lipid content of the dry matter content of the fermentation broth is at least 40 wt. %.

Methods for producing a fermentation broth are known in the art (see, e.g., WO 91/07498, WO 94/08467 and WO 97/37032). Generally, fermentation broths are produced by culturing microbial cells in a fermenter in the presence of a carbon source and a nitrogen source, along with additional substances that facilitate growth of microbial cells. Once a desired biomass density has been reached, lipid production may be induced by various measures. Possible measures include limiting the nitrogen source, limiting the carbon source, limiting the oxygen content in the fermenter, and combinations thereof.

Once a desired lipid content has been reached (i.e., a total lipid content of at least 40 wt. %), the fermentation may be stopped and the viscosity of the fermentation broth reduced using the method according to the invention. This method leads to a reduction of the viscosity of a fermentation broth (relative to the viscosity of the fermentation broth (immediately) before addition of the at least one compound). The method may lead to a relative viscosity reduction of the fermentation broth of at least 10%, preferably at least 15%, more preferably at least 20%, still more preferably at least 25%, still more preferably at least 35%, more preferably at least 40%. As a maximum, a relative viscosity reduction of the fermentation broth of 90% may be mentioned. The relative viscosity reduction can be determined by measuring the viscosity of the fermentation broth before and after addition of the at least one compound by measurement at a shear rate of 9.8-10.0 s⁻¹, 25° C., and according to a shear rate logarithmic ramp up and down isothermal sweep (shear rate range 0.1-100 s⁻¹) using a Malvern Kinexus Pro+ rheometer with a cup and bob system. To determine the viscosity difference, the viscosity of the fermentation broth should be measured immediately (e.g., at most 1 min.) before addition of the at least one compound and 15 minutes after addition of the at least one compound to the fermentation broth.

At least one compound selected from the group consisting of ascorbic acid, citric acid, lactic acid, formic acid, amino acids, and soluble salts of said acids is added to the fermentation broth to reduce the viscosity of the fermentation broth. “Amino acids” as used herein refer to added amino acids, i.e. amino acids that are added to a fermentation broth. Amino acids particularly suitable for reducing the viscosity of a fermentation broth are natural amino acids (both L- and D-stereoisomers), in particular amino acids selected from the group consisting of histidine, cysteine, methionine, lysine, glutamic acid, and glutamine and their salts. In a preferred method, the at least one compound is selected from the group consisting of ascorbic acid, citric acid, lactic acid, formic acid, and soluble salt of said acids (calculated on the total weight of the microbial cells). Preferred compounds are ascorbic acid, citric acid, lactic acid, formic acid, and soluble salts of said acids. Ascorbic acid and soluble salts thereof are particularly preferred compounds. This is because ascorbic acid, citric acid, lactic acid, and soluble salts thereof were found to result in the most significant relative viscosity reductions (relative viscosity reductions were greater than 35%, usually greater than 40%). The soluble salt may, e.g., have a metallic cation or an ammonium cation. The soluble salt may, e.g., be a (di) sodium salt, a magnesium salt, a calcium salt, a (di) potassium salt, a (di) lithium salt, or an ammonium salt. The preferred soluble salts are sodium ascorbate, magnesium ascorbate, calcium ascorbate, sodium citrate and magnesium citrate. The soluble salts generally dissolve (or remain dissolved when added as a solution) in the fermentation broth after being mixed thereinto. The soluble salts may have a solubility in water (at 20° C.) of at least 0.01 g/mL, preferably 0.03 to 2.0 g/mL, more preferably 0.05 to 1.0 g/mL. In addition to the at least one compound, a soluble inorganic salt (for example a sulfate salt, like as iron sulfate, sodium sulfate, magnesium sulfate, copper sulfate, aluminum sulfate, and calcium sulfate) or a hydrate thereof may be added. The at least one compound may be added to the fermentation broth as a solid or as a solution, e.g. an aqueous solution.

The total amount of compound mixed into the fermentation broth may be at least 0.05 wt. % to at most 10 wt. % (calculated on the total weight of the fermentation broth). The total amount of compound mixed into the fermentation broth is preferably at least 0.07 wt. % to at most 8.0 wt. %, more preferably at least 0.10 wt. % to at most 7.0 wt. %, more preferably at least 0.12 wt. % to at most 6.0 wt. %, still more preferably at least 0.15 wt. % to at most 5.0 wt. %. Each compound may independently be mixed into the fermentation broth in an amount of 0.05 wt. % to 5.0 wt. % (calculated on the total weight of the fermentation broth), preferably in an amount of 0.10 wt. % to at most 4.0 wt. %, more preferably in an amount of 0.15 wt. % to 3.0 wt. %. For example, if a combination of citric acid and lactic acid (or soluble salts thereof) are mixed into the fermentation broth, citric acid (or a salt thereof) and lactic acid (or a salt thereof) may each, independently, be mixed into the fermentation broth in an amount of 0.15 wt. % to 3.0 wt. %.

The at least one compound may be mixed into a fermentation broth at any suitable time point, for example, at the end of the fermentation process or during the fermentation process. For example, the at least one compound may be mixed into the fermentation broth when the fermentation broth's cell dry weight is at least 70 g/kg, preferably at least 90 g/kg, more preferably at least 110 g/kg, even more preferably at least 130 g/kg, still more preferably at least 145 g/kg, still more preferably at least 160 g/kg, still more preferably at least 180 g/kg during the fermentation process. As a maximum, the cell dry weight of the fermentation broth may be at most 300 g/kg when the at least one compound is mixed into the fermentation broth. In another example, at least one compound may be added to a fermentation broth after completion of the fermentation process. The dry cell weight of the fermentation broth is determined by washing the broth with deionized water and determining the total dry matter of the obtained sample, as is well-known to the skilled person.

The mixing time (following addition of the at least one compound to the fermentation broth) may be from 1 minute to 1 hour, preferably from 3 to 30 minutes, preferably from 5 to 20 minutes. Mixing helps to achieve a uniform viscosity reduction throughout the fermentation broth. However, the mixing time is preferably as short as possible. This is because mixing may cause some cells to lyse and release their microbial oil, which would likely negatively affect the viscosity reduction of the fermentation broth. For similar reasons, it is preferred that the mixing elements used for the mixing are static mixing elements (e.g., rods, plates), even though dynamic mixing elements can also be used.

The at least one compound may be mixed into a fermentation broth comprising 5.0 wt. % or less of free microbial oil (calculated on the total weight of the fermentation broth). In other words, the fermentation broth may comprise 5.0 wt. % or less of free microbial oil when the at least one compound is mixed into the fermentation broth. Such fermentation broths contain a low amount of lysed microbial cells. The amount of free microbial oil in the fermentation broth (i.e., the free microbial oil content of the fermentation broth) is preferably 4.0 wt. % or less, preferably 3.0 wt. % or less, more preferably 2.0 wt. % or less, more preferably 1.0 wt. % or less, more preferably 0.9 wt. % or less, more preferably 0.8 wt. % or less, still more preferably 0.6 wt. % or less, still more preferably 0.6 wt. % or less, still more preferably 0.5 wt. % or less. An advantage associated with a low amount of free microbial oil in the fermentation broth is that further processing is significantly simplified, as there is no impact on drying capacity, dry biomass agglomeration during conveying, or oil quality.

For the same reason, it is also desirable that (substantially) no free microbial oil is released after the at least one compound has been added to the fermentation broth. The amount of free microbial oil in the fermentation broth, 15 minutes after addition of the at least one compound, may be 10.0 wt. % or less, preferably 9.0 wt. % or less, more preferably 8.0 wt. % or less, more preferably 7.0 wt. % or less, more preferably 6.0 wt. % or less, preferably 5.0. More preferably, the amount of free microbial oil in the fermentation broth, 15 minutes after addition of the at least one compound, is not higher than the amount of free microbial oil immediately before addition of the at least one compound. It is an advantage of the present invention that, even with microbial cells having a relatively high oil content, the free oil content in the fermentation broth can be maintained below a predetermined value.

The free microbial oil content in the fermentation broth is determined as follows. Fermentation broth is contacted with isopropyl alcohol at 70° C. and centrifuged for 5 minutes at 12,000 rpm. Then, hexane (solvent) and water are added to a portion of the separated supernatant, mixed and subjected to centrifugation at 12,000 rpm for 1 minute. The resulting solvent/free microbial oil partition is transferred to a tray, which is placed in a fume hood. Hexane is evaporated at room temperature until stable, leaving only the free microbial oil, the amount of which can be determined gravimetrically.

The water content of the fermentation broth may, for example, be at most 90 wt. % (calculated on the total weight of the fermentation broth), preferably at most 85 wt. %. The water content of the fermentation broth may be greater than 10 wt. %, preferably at least 20 wt. %, more preferably at least 30 wt. %, more preferably at least 50 wt. %, more preferably at least 75 wt. %. The water content is determined gravimetrically, in particular using a halogen balance at 120° C., e.g. by distributing 1.8 g of broth equally over a glass fiber filter and incubating at 120° C. in a Mettler Toledo moisture analyzer.

The fermentation broth contains dry matter (i.e., insoluble components), such as microbial cells and free microbial oil. Other dry matter that may be present in the fermentation broth may be lysed microbial cells and optional other components, such as salts. The ‘dry matter content’ is defined as follows:

$$Dry matter content ( wt . \% ) = 100 \% - water content ( wt . \% )$$

The dry matter content of the fermentation broth may be at least 10 wt. %, preferably at least 11 wt. %, more preferably at least 12 wt. %, still more preferably at least 13 wt. %, still more preferably at least 14 wt. %, still more preferably at least 15 wt. % (calculated on the total weight of the fermentation broth). The dry matter content is preferably at most 25 wt. %, more preferably at most 24 wt. %, still more preferably at most 23 wt. %, still more preferably at most 22 wt. %, still more preferably at most 21 wt. %, still more preferably at most 20 wt. %.

The microbial cells in the fermentation broth may be oleaginous yeast cells (e.g., Yarrowia lipolytica, Rhodotorula glutinis, Cryptococcus curvatus or Lipomyces starkeyi), fungal cells, or microalgal cells, preferably microalgal cells. The oleaginous microalgal cells may be from the phylum Stramenopiles, particularly from the taxon Labyrinthulomycetes, more particularly from the family of Thraustochytriaceae. The microalgal cells in these families produce significant amounts of DHA, which is a valuable dietary ingredient. Microalgal cells from the family Thrausochytriaceae are preferably from the genus Schizochytrium (such as S. aggregatum), Thraustochytrium (such as T. aggregatum, T. multirudimentale, and T. striatum), Aurantiochytrium (such as A. limacinum and A. mangrovei), or Ulkenia (such as U. visurgensis and U. amoeboidea). Other preferred groups of microalgae include microalgae from the family of Crypthecodiniaceae, particularly from the genus Crypthecodinium.

The microbial oil may comprise 30 to 90 wt. % of polyunsatured fatty acids (PUFAs) (calculated on the total weight of fatty acids in the total lipid content, e.g. as determined by FAME analysis), preferably 40 to 85 wt. %, more preferably 50 to 80 wt. %, more preferably 55 to 75 wt. %, optionally of PUFAs comprising three or more double bonds. The microbial oil may comprise 10 to 80 wt. % of DHA (calculated on the total weight of fatty acids in the total lipid content, e.g. as determined by FAME analysis), preferably 20 to 75 wt. %, more preferably 30 to 70 wt. %, more preferably 50 to 65 wt. %. The microbial oil may also comprise 5.0 to 25 wt. % of docosapentaenoic acid (DPA) (based on the total weight of fatty acids in the total lipid content, e.g. as determined by FAME analysis), preferably 10 to 24 wt. %, more preferably 12 to 22 wt. %, still more preferably 14 to 20 wt. %. The microbial oil may also comprise 0.1 to 5.0 wt. % of eicosapentaenoic acid (EPA) (based on the total weight of fatty acids in the total lipid content, e.g. as determined by FAME analysis), preferably 0.2 to 4.0 wt. %, more preferably 0.3 to 3.0 wt. %. The microbial oil may, for example, comprise DHA (in the amounts as defined above) and EPA (in the amounts as defined above). The microbial oil may comprise DHA, EPA, and DPA (all in the amounts as defined above).

As a result of mixing the at least one compound into the fermentation broth, the pH of the fermentation broth may decrease or increase (i.e., the fermentation broth may become more acidic or alkaline).

In embodiments wherein the fermentation broth becomes more acidic through the addition of the at least one compound, the pH of the fermentation broth (e.g. in the range of pH 4.0 to pH 7.5) may decrease by at least 0.1 units on the pH scale relative to the pH of the fermentation broth (immediately) before the at least one compound is mixed into the fermentation broth. It is preferred that the pH decreases by at least 0.3 units on the pH scale relative to the pH of the fermentation broth (immediately) before the at least one compound is mixed into the fermentation broth, more preferably at least 0.5 units, even more preferably at least 1.0 units, and optionally up to 3.0 units, more preferably up to 2.5 units. For example, the resulting fermentation broth (after addition of the at least one compound) may have a pH of 5.0 or less, for example a pH of from 2.0 to 4.0.

In these embodiments, the original pH of the fermentation broth (i.e., the pH of the fermentation broth before the at least one compound was added) may be at least partially restored by the addition of a base. This may, for example, be a base that is free from chloride atoms, as this helps prevent corrosion of the reactor. For example, the base may be a strong organic or inorganic base, preferably a hydroxide salt, preferably an alkali hydroxide, more preferably an alkali hydroxide selected from the group consisting of lithium hydroxide, sodium hydroxide, and potassium hydroxide. After addition of the base, the pH of the fermentation broth may increase (relative to the pH of the fermentation broth after addition of the at least one compound) by at least 0.1 units on the pH scale, preferably at least 0.3 units, more preferably at least 0.5 units, even more preferably at least 1.0 units, and up to 3.0 units, more preferably up to 2.5 units. Accordingly, after addition of the base, the pH of the fermentation broth may be greater than 4.0 (for example, to a pH of greater than 4.0 to 7.0, preferably to a pH of 4.5 to 6.5).

In embodiments wherein the fermentation broth becomes more alkaline through the addition of the at least one compound, the pH of the fermentation broth (e.g. in the range of pH 3.0 to pH 5.5) may increase by at least 0.1 units on the pH scale relative to the pH of the fermentation broth (immediately) before the at least one compound is mixed into the fermentation broth. The pH of the fermentation broth may increase by at least 0.3 units on the pH scale relative to the pH of the fermentation broth (immediately) before the at least one compound is mixed into the fermentation broth, optionally by at least 0.4 units, and optionally up to 1.0 units, more preferably up to 0.7 units, even more preferably up to 0.6. For example, the resulting fermentation broth (after addition of the at least one compound) may have a pH of 5.0 or more, for example a pH of from 5.0 to 6.0.

In these embodiments, the original pH of the fermentation broth (i.e., the pH of the fermentation broth before the at least one compound was added) may be at least partially restored by the addition of an additional acid. The additional acid for restoring the pH may be an acid that is free from chloride atoms, as this helps prevent corrosion of the reactor. The additional acid may be a strong organic or inorganic acid, preferably a strong inorganic acid, more preferably sulfuric acid, phosphoric acid, or nitric acid. After addition of the additional acid, the pH of the fermentation broth may decrease (relative to the pH of the fermentation broth after addition of the base) by at least 0.1 units on the pH scale, preferably at least 0.3 units, more preferably at least 0.5 units, even more preferably at least 1.0 units, and up to 3.0 units, more preferably up to 2.5 units. Accordingly, after addition of the additional acid, the pH of the fermentation broth may decrease to a pH of 5.5 or less (for example, to a pH of 3.0 to 5.5, preferably to a pH of 4.0 to 5.0).

It has been found that the resulting “pH swing” (i.e., decreasing the pH of the fermentation broth from an initial pH, followed by re-adjusting the pH of the fermentation broth to make the fermentation broth more alkaline, or vice versa) can surprisingly lead to an even greater relative viscosity reduction. When re-adjusting the pH of the fermentation broth, it is possible to re-adjust the pH only slightly, so that the adjusted pH is lower than the original pH of the fermentation broth. It is also possible to re-adjust the pH such that the original pH of the fermentation broth is restored. Finally, it is possible to re-adjust the pH so that the pH of the fermentation broth is more acidic or more alkaline (whichever is applicable) than the original pH of the of the fermentation broth. For example, one could decrease the pH of a fermentation broth from pH 5.0 to pH 4.5 by addition of at least one compound and, subsequently, increase the pH from pH 4.5 to pH 4.7 (partial restoration of the original pH), pH 5.0 (restoration of the original pH), or pH 6.0 (re-adjustment to overshoot the original pH) by the addition of a base.

Thus, also disclosed herein is a preferred method for reducing the viscosity of a fermentation broth, wherein the method comprises the steps of
mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated the total weight of the fermentation broth, wherein the total lipid content of the dry matter content of the fermentation broth is at least 40 wt. %, thereby decreasing or increasing the pH of the fermentation broth (e.g., by 0.1 to 3.0 units on the pH scale); and
re-adjusting the pH of the fermentation broth in the opposite direction (i.e., increasing or decreasing the pH, respectively) by addition of a base or an acid.

In order to facilitate the pH swing, it may be advantageous to further decrease (if the pH of the fermentation broth decreased after the addition of the at least one compound) or further increase (if the pH of the fermentation broth increased after the addition of the at least one compound) by the addition of a further acid or further base, respectively. This was found to be particularly advantageous in cases where the pH of the fermentation broth decreased after the at least one compound was mixed thereinto. The further acid may be added to the fermentation broth in order to further decrease the pH of the fermentation broth, e.g. to a pH of from 2.0 to 4.0 (when measured at room temperature of about 25° C.). The further acid may be a chloride-free acid (such as, but not limited, to sulfuric acid, phosphoric acid, nitric acid or organic acids, such as, but not limited to, oxalic acid and lactic acid). It is noted that adding a further acid can even be advantageous if a pH swing is not performed. Indeed, as shown in the Examples, it has been found that further reducing the pH of the fermentation broth with an further acid surprisingly amplifies the relative viscosity reduction resulting from the at least one compound. The further base may be added to the fermentation broth in order to further increase the pH of the fermentation broth, e.g. to a pH of from 5.5 to 7.5 (when measured at room temperature of about 25° C.). The further base may be a chloride-free base, such as a hydroxide salts (e.g. lithium hydroxide, sodium hydroxide, potassium hydroxide, magnesium hydroxide).

Thus, also disclosed herein is a preferred method for reducing the viscosity of a fermentation broth, wherein the method comprises the steps of
mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated the total weight of the fermentation broth, wherein the total lipid content of the dry matter content of the fermentation broth is at least 40 wt. %; and
adding a further acid or base to the fermentation broth.

The period of time during which the fermentation broth is at a particular pH is preferably controlled. This is because microbial cells may lyse e.g. if the pH of the fermentation broth is kept at a low pH for too long. As explained above, lysis of microbial cells would likely be detrimental to the viscosity reduction achieved with the inventive method. Therefore, it is preferred that the altered pH conditions (i.e., the pH after addition of the at least one compound and, optionally, the further acid or base) is maintained for at most 1 hour, preferably at most 45 minutes, more preferably at most 30 minutes. The altered pH conditions may be maintained for at least 1 minute, preferably at least 3 minutes, more preferably at least 5 minutes, in order to achieve a good viscosity reduction. If a pH swing is performed, the re-adjusted pH conditions achieved by adding a base to the fermentation broth may also be maintained from 1 minute to 1 hour, preferably from 3 minutes to 45 minutes, more preferably from 5 minutes to 30 minutes.

It may be desirable to also control the temperature of the fermentation broth, as the temperature of the fermentation broth could also affect the rate of cell lysis. It is within the scope of the skilled person to take the necessary actions to avoid cell lysis. For example, the fermentation broth, immediately (e.g., at most 1 min.) before the at least one compound is mixed thereinto, may have a temperature of at most 35° C., preferably at most 30° C., more preferably at most 25° C. When the temperature of the fermentation broth is too low, this may e.g. lead to an increased viscosity of the fermentation broth. Therefore, the temperature of the fermentation broth, immediately (e.g., at most 1 min.) before the at least one compound is mixed thereinto, may be at least 5° C., more preferably at least 10° C. It will be evident to the skilled person that a temperature as defined above can be maintained for as long as a fermentation broth having a viscosity is desired. Of course, the skilled person would understand that the low temperature of the fermentation broth need not be maintained indefinitely; the temperature can, for example, be increased during an evaporation or drying step, as will be discussed below.

After the steps for reducing the viscosity have been performed (i.e., after mixing the at least one compound into the fermentation broth and, optionally, additional acid and/or performing the optional pH swing), the fermentation broth may have a viscosity of at most 1.0 Pa·s. The fermentation broth preferably has a viscosity of at most 0.5 Pa·s, more preferably at most 0.2 Pas, still more preferably at most 0.1 Pa·s. The fermentation broth, after the viscosity has been reduced, may have a viscosity of at least 0.001 Pa·s, preferably a viscosity of at least 0.01 Pa·s. Such a low viscosity is desirable, as viscosity has conventionally been a limiting factor in the downstream processing of fermentation broths. When the viscosity of the fermentation broth is reduced as disclosed herein, the viscosity of the resulting fermentation broth is such that it can readily be processed in an evaporator. Importantly, the low viscosity of the resulting fermentation broth allows an increase of the amount of dry matter sent to the evaporator at a given time. This, in turn, results in substantial increases in process efficiency.

Fermentation Broth with a Low Viscosity

Also disclosed herein is a fermentation broth comprising microbial cells, at least one compound selected from the group consisting of ascorbic acid, citric acid, lactic acid, formic acid, amino acids, and soluble salts thereof, and 5.0 wt. % or less of free microbial oil (calculated on the total weight of the fermentation broth), wherein the total lipid content of the dry matter content of the fermentation broth is at least 40 wt. %, optionally wherein the fermentation broth has a viscosity of 1.0 Pa·s or less, measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

The preferences specified for the fermentation broth according to the invention equally apply to the fermentation broth with a low viscosity defined above.

Methods for Processing the Fermentation Broth with a Low Viscosity

Also disclosed herein are methods for processing a fermentation broth with a low viscosity, such as the fermentation broths according to the invention (e.g., as obtained by a “Method for reducing the viscosity of a fermentation broth” discussed above). Any preferences for the “Method for reducing the viscosity of a fermentation broth” and a fermentation broth according to the invention equally apply to the methods for processing a fermentation broth disclosed herein.

Methods for processing a fermentation broth obtained by the method for reducing the viscosity of a fermentation broth according to the invention or a fermentation broth according to the invention may comprise one of the following process sequences:
(a) concentrating the fermentation broth (e.g. by evaporation (of water), filtration, or centrifugation) to obtain a concentrated fermentation broth;
(b) concentrating the fermentation broth (e.g. by evaporation (of water), filtration, or centrifugation) to obtain a concentrated fermentation broth, and, thereafter, drying the concentrated fermentation broth to obtained a dried biomass;
(c) concentrating the fermentation broth (e.g. by evaporation (of water), filtration, or centrifugation) to obtain a concentrated fermentation broth, drying the concentrated fermentation broth to obtained a dried biomass, and lysing the dried biomass (optionally in the presence of oil) to obtain a dispersion comprising lysed microbial cells in oil;
(d) concentrating the fermentation broth (e.g. by evaporation (of water), filtration, or centrifugation) to obtain a concentrated fermentation broth, lysing microbial cells in the concentrated fermentation broth to obtain a concentrated fermentation broth comprising lysed microbial cells, and separating microbial oil from the concentrated fermentation broth comprising lysed microbial cells in one or more steps, optionally without performing a demulsification step (e.g., adding an additional demulsification agent) before separating the microbial oil;
(e) drying the fermentation broth (without first concentrating the fermentation broth) to obtain a dried biomass;
(f) drying the fermentation broth (without first concentrating the fermentation broth) to obtain a dried biomass and, thereafter, lysing the dried biomass (optionally in the presence of oil) to produce a dispersion comprising lysed microbial cells in oil;
(g) lysing microbial cells in the fermentation broth to obtain a fermentation broth comprising lysed cells;
(h) lysing microbial cells in the fermentation broth to obtain a fermentation broth comprising lysed cells and separating microbial oil from the fermentation broth comprising lysed cells in one or more steps, optionally wherein a demulsification step is performed prior to separating microbial oil from the fermentation broth comprising lysed cells; or
(i) lysing microbial cells in the fermentation broth to obtain a fermentation broth comprising lysed cells, concentrating the fermentation broth comprising lysed cells (e.g., by evaporation (of water) or centrifugation), and separating microbial oil from the fermentation broth comprising lysed cells in one or more steps, optionally wherein a demulsification step is performed prior to separating microbial oil from the fermentation broth comprising lysed cells.

### Process Sequences (a) to (d)

Process sequences (a) to (d) all involve a first step of concentrating the fermentation broth with a reduced viscosity to obtain a concentrated fermentation broth. As will be understood by the skilled person, the fermentation broth is concentrated by the removal of a portion of the water from the fermentation broth. The concentrated fermentation broth obtained in these process sequences appears to have an improved flowability as compared concentrated fermentation broths not comprising the at least one compound.

Suitable methods for concentrating the fermentation broth include evaporation (e.g., falling film evaporation, wiped film evaporation, or combinations thereof), filtration and centrifugation. Evaporation, in particular falling film evaporation and/or wiped film evaporation, is a preferred method for concentrating the fermentation broth. When falling film evaporation is used to concentrate the fermentation broth, water may be evaporated from the fermentation broth at a temperature of from 35 to 90° C., preferably from 40 to 85° C., more preferably from 45 to 80° C., even more preferably from 50 to 75° C. When wiped film evaporation is used to concentrate the fermentation broth, water may be evaporated from the fermentation broth at a temperature of from 35 to 90° C., preferably from 40 to 85° C., more preferably from 45 to 80° C., even more preferably from 50 to 75° C. Lower evaporation temperatures may be preferred (also in the other methods for concentrating the fermentation broth mentioned above), as lower temperatures are less destructive to the unsaturated fatty acids (such as DHA) in the microbial oil present in the microbial cells.

The amount of water removed during the concentration step may be expressed as a concentration factor. The concentration factor (calculated on the volume of the fermentation broth) may be 1.2 to less than 3.0, preferably 1.3 to 2.5, more preferably 1.4 to 2.0, even more preferably 1.4 to 1.8. The concentrated fermentation broth may comprise from 40 wt. % to 80 wt. % of water (calculated on the total weight of the fermentation broth), preferably from 60 to 75 wt. %, more preferably from 65 to 70 wt. %.

Once a concentrated fermentation broth has been obtained, the concentrated fermentation broth may be subjected to a drying step, as in process sequences (b) and (c). It is advantageous to concentrate the fermentation broth with a reduced viscosity by means of a concentration step prior to subjecting it to a drying step, as this reduces the minimum residence time in the dryer and so allows for a higher throughput.

In this drying step, water is (further) removed from the concentrated fermentation broth, thereby resulting in a dried biomass. As used herein, a “dried biomass” refers to material obtained by drying the fermentation broth to a water content of 5 wt. % or less, wherein the fermentation broth contains solids (i.e., insoluble components), such as microbial cells comprising lipids, lysed cells, other cell matter, free oil, media salts, optionally one or more compounds added to the fermentation broth. Drying of the concentrated fermentation broth may, for example, be done by means drum drying, pneumatic drying, spray drying, and/or freeze drying. Spray drying may be accomplished by a box-dryer, a tall-form spray-dryer, a fluidized bed dryer, and/or a moving fluidized bed dryer (e.g., a FilterMat® spray-dryer, GEA Process Engineering, Inc.). It should be noted that all of these drying methods suffer from the fact that they are not particularly suitable for drying fermentation broths having a high dry matter content (e.g., fermentation broths having a high microbial oil content), because fermentation broths having a high dry matter content are usually too viscous to be dried using these drying methods. Because the method for reducing the viscosity according to the invention yields fermentation broths with a reduced viscosity, the concentrated fermentation broths obtained according to process sequences (a) to (c) can be processed using these drying methods, even when their dry matter content is high (i.e., exceeding 25 wt. %).

Drying of the concentrated fermentation broth may lead to a concentration factor (calculated on the volume of the concentrated fermentation broth) of at least 2.0, preferably at least 2.5, more preferably at least 3.0. As a maximum, a value of concentration factor of 5.0 may be mentioned for the concentrated fermentation broth. The dried biomass obtained after drying may comprise 4 wt. % or less of water (calculated on the total weight of the dried biomass), preferably 3 wt. % or less, more preferably 2 wt. % or less, more preferably 1 wt. % or less, even more preferably 0.5 wt. % or less. As a minimum, the dried biomass may comprise 0.001 wt. % of water (calculated on the total weight of the dried biomass). In some embodiments, the dried biomass comprises at least 0.01 wt. % of water, optionally 0.1 wt. % of water. Other features of the dried biomass obtained will be discussed below, under the header “Dried biomass.”

The drying step may be followed by a step of lysing the dried biomass. Methods suitable for lysing microbial cells in a dried biomass are known in the art. An advantage of performing a drying step prior to cell lysis is that the volume of the stream reduces during the drying step, thereby allowing for the use of small (and, thus, more economical) equipment in downstream processing steps.

In preferred process sequence (c), the drying step is followed by lysing the dried biomass (optionally in the presence of oil) to obtain a dispersion of lysed microbial cells in oil. Microbial cells in the dried biomass may be lysed in the presence of oil by mechanical means, such as a high pressure homogenizer. As an example, lysis of microbial cells can be achieved by passing the broth through a high shear homogenizer for 2-80 passes and at pressures higher than 300 bar.

Oils suitable to be used in process sequence (c) (and process sequence (d), which is discussed in more detail below) include oils such as microbial oil, fish oil, and/or oil from a plant material. The oil may be used in crude, unfiltered form or may be used in any modified supply form (e.g., degummed and/or refined). The oil derived from a plant material may be derived from coconut, corn, cottonseed, olive, palm, peanut, walnut, rapeseed, canola, safflower, sesame, soybean, sunflower, flaxseed, linseed, camelina, shea, or citrus, or one or more combinations thereof. The fish oil may be oil derived from a krill or a fish selected from herring, menhaden, anchovy, pilchard, sardine, or mackerel, tuna, or one or more combinations thereof. The microbial oil may be extracted from microbial cells that are different from the source of microbial cells used to make a lysed cell in oil suspension. For example, Schizochytrium cells may be lysed in the presence of in oil extracted from Crypthecodinium cells. Microbial oil from the same organism can also be used to make a suspension. For example, Schizochytrium cell matter may be lysed in the presence of oil extracted from Schizochytrium cells to produce a lysed-cell-in-oil suspension. By lysing dried biomass in the presence of microbial oils, amounts of valuable biomaterials (such as arachidonic acid (ARA), EPA carotenoids, or astaxanthin) can be optimized. The microbial oils defined above may also be added to a (lysed) dried biomass, as will be discussed below.

If the cell lysis of the dried biomass takes place in the presence of an oil, as in process sequence (c), the biomass-to-oil ratio may be from 1:10 to 10:1, preferably 3:10 to 9:1, more preferably 5:10 to 8:1, even more preferably 1:1 to 7:1, even more preferably 2:1 to 6:1, still more preferably 3:1 to 5:1.

The product obtained by process sequence (c) is a dispersion comprising lysed cells in oil, which may be used in a variety of applications, but is normally used to formulate animal feeds, in particular fish feeds, more in particular salmon feeds.

In process sequence (d), concentration of the fermentation broth as described above is followed by lysis of microbial cells in the concentrated fermentation broth. This yields a concentrated fermentation broth comprising lysed microbial cells, and releases microbial oil from the microbial cells, which can be separated in one or more steps.

Aside from cell lysis by mechanical means, as described above in the context of process sequence (c), microbial cells in the concentrated fermentation broth obtained in process sequence (d) may be lysed by means of enzymatic cell lysis, e.g. using a protease (e.g., an endopeptidase, an exopeptidase, a trypsin, a pepsin), a lysozyme, and/or a nuclease. For example, cell lysis may be achieved by lysing concentrated fermentation broth with 0.005 to 0.5 wt. % of a serine endopeptidase (e.g., Alcalase®) on the weight of the concentrated fermentation broth, preferably at a pH of from pH 7.0 to 9.0 (preferably, from pH 7.5 to 8.0). The concentrated fermentation broth may be exposed to the enzyme for a period of from 2 to 36 hours, preferably 3 to 21 hours, more preferably a period of from 4 to 16 hours and optionally at a temperature of from 55 to 70° C., preferably from 60 to 65° C.

The microbial oil may be separated in one or more steps from the fermentation broth comprising lysed cells obtained in the last step of process sequence (d). For example, microbial oil can be separated by means of a mechanical solid-liquid separation, such as centrifugation or filtration. Additionally or alternatively, the microbial oil can be extracted from the fermentation broth. Suitable extraction methods are described in, e.g., WO 2001/076385 and WO 2001/076715. If so desired, extraction can be performed such that a specific lipid fraction is separated. Polar lipids or polar lipid containing mixtures may be extracted using a polar organic solvent (e.g., an alcohol) from a lysed broth, while non-polar lipids may be extracted using a non-polar organic solvent (e.g., hexane).

It has surprisingly been found that, after the lysis step required by process sequence (d), demulsification may not be required; an oil layer may readily form and then be separated (e.g. extracted) from the cellular debris and the aqueous layer. This is in contrast to processes known in the art, in which an oil layer generally does not form without the addition of a demulsification agent.

### Process Sequences (e) and (f)

Process sequences (e) and (f) are processes wherein the fermentation broth with a reduced viscosity is dried, without having been subjected to a concentration step. A drying step removes more water than a concentration step, as will be understood by a person skilled in the art.

An advantage associated with process sequences (e) and (f) is that the drying step results in a (more) consistently dried biomass than when a viscous fermentation broth is dried with the same drying step. This is because the distribution of a viscous fermentation broth over a dryer varies more and so is generally uneven.

The fermentation broth (i.e., the fermentation broth having a reduced viscosity) may be dried by means of one or more drying steps, which result in the removal of water from the fermentation broth. Water may be removed by means of falling film evaporation, wiped film evaporation, or combinations thereof. Additionally or alternatively, water can be removed by drum drying, pneumatic drying, spray drying, and/or freeze drying. Spray drying can be accomplished by a box-dryer, a tall-form spray-dryer, a fluidized bed dryer, or a moving fluidized bed dryer (e.g., a FilterMat® spray-dryer, GEA Process Engineering, Inc.). Preferences for each of these evaporation and drying methods have been discussed above in the context of process sequences (a) to (c) and these preferences also apply to methods requiring any one of process sequences (e) and (f).

Drying of the fermentation broth may lead to a concentration factor (calculated on the volume of the fermentation broth) of at least 3.0, preferably at least 4.0, more preferably at least 5.0. The dried biomass obtained after evaporation or drying may comprise 5 wt. % or less of water (calculated on the total weight of the dried biomass), preferably 3 wt. % or less, more preferably 2 wt. % or less, more preferably 1 wt. % or less. As a minimum, the dried biomass may comprise 0.001 wt. % of water (calculated on the total weight of the dried biomass). As mentioned above in the context of process sequences (a) to (d), other features of the dried biomass obtained will be discussed below, under the header “Dried biomass.”

As mentioned above, the drying methods defined above suffer from the fact that it is difficult to dry fermentation broths having a high dry matter content (e.g., fermentation broths having a high microbial oil content), because fermentation broths having a high dry matter content are usually too usually viscous to be dried using these drying methods. However, because the fermentation broth (obtained) according to the invention has a low viscosity, it can have a higher dry matter content when it is dried, as also mentioned above.

The dried biomass obtained by drying the fermentation broth with a reduced viscosity may be lysed, resulting in a dispersion of lysed microbial cells in microbial oil. Preferences for this cell lysis step are defined above in the context of process sequence (c). It is preferred that cell lysis of the dried biomass is achieved by means of mechanical cell disruption, e.g. as described in WO 2018005856 and WO 2020141206. If so desired, the microbial oil may be separated in one or more steps from the lysed microbial cells by any suitable method, for example, by means of filtration or decantation. Preferences for the separated microbial oil are defined under the header “Microbial oil” below and in the context of the method for reducing the viscosity of a fermentation broth according to the invention.

The dispersion of lysed microbial cells in microbial oil may be diluted with another oil. Suitable oils include microbial oil, fish oil, and/or oil from a plant material. The oil may be used in crude, unfiltered form or may be used in any modified supply form (e.g., degummed and/or refined). The oil derived from a plant material may be derived from coconut, corn, cottonseed, olive, palm, peanut, walnut, rapeseed, canola, safflower, sesame, soybean, sunflower, flaxseed, linseed, camelina, shea, or citrus, or one or more combinations thereof. The fish oil may be oil derived from a krill or a fish selected from herring, menhaden, anchovy, pilchard, sardine, or mackerel, tuna, or one or more combinations thereof. The microbial oil may be extracted from microbial cells that are different from the source of microbial cells used to make a dried biomass comprising lysed microbial cells. It may also be an oil from the same organism (i.e., the same microbial cell).

In process sequence (e), the dried biomass obtained by drying the fermentation broth with a reduced viscosity is lysed in the presence of an oil to form a dispersion of lysed microbial cells in oil. Preferences for this cell lysis step are defined above in the context of process sequence (c). It is preferred that cell lysis of the dried biomass is achieved by means of mechanical cell disruption, e.g. as described in WO 2018005856 and WO 2020141206.

### Process Sequences (g) to (i)

In process sequences (g) to (i), the fermentation broth with a reduced viscosity is lysed to obtain a fermentation broth comprising lysed cells. The fermentation broth comprising lysed cells generally further comprises microbial oil and water. Lysis may be achieved by a variety of methods, including (but not limited to) enzymatic cell lysis, a combination of pH modification and/or temperature, mechanical lysis (e.g. high shear homogenization). Suitable methods are described in the context of process sequences (a) to (c) above.

An advantage associated with process sequences (g) to (i) is that, because the fermentation broth has a reduced viscosity, less power is required to lyse the cells than would be required for viscous fermentation broths lysed using the same cell lysis method.

In process sequence (h), the microbial oil is separated from the fermentation broth comprising lysed cells in one or more steps. This may be done by means of a solid-liquid separation, such as a centrifuge or a filter.

In process sequence (i), the fermentation broth comprising lysed cells is concentrated. Concentration of the fermentation broth may be done using, e.g., evaporation (of water) or centrifugation. Suitable methods are described above in the context of process sequences (a) to (c).

It may not be necessary to add an additional demulsification agent to the fermentation broth comprising lysed cells in order to achieve phase separation (i.e., a partitioning of the oil phase comprising microbial oil and the aqueous phase comprising water), which may be an advantage over processes known in the art.

In some embodiments, a demulsification step (e.g. adding a demulsification agent) is performed prior to separating microbial oil from the fermentation broth comprising lysed cells. Methods for demulsifying a fermentation broth comprising lysed cells, microbial oil, and water are known in the art.

As mentioned above, the evaporation and drying methods defined above are greatly facilitated by the low viscosity of the fermentation broth obtained according to the invention. Concentrated fermentation broth with higher dry matter content can be obtained and processed, because of the reduced viscosity. In addition, a more energy efficient water removal process is obtained, as less water needs to be removed by drying to get to dry biomass. This means higher throughputs can be obtained.

### Microbial Oil

Microbial oil isolated from the microbial cells in the fermentation broth, e.g. using any of the methods defined above, has a surprisingly good oxidative stability, even as a crude microbial oil.

The crude microbial oil may be subjected to one or more purification steps known in the art. For example, the crude microbial oil may be subjected to distillation or urea adduction to produce a product with higher concentrations of DHA or another fatty acids. Additionally or alternatively, the crude microbial oil may be subjected to winterization, degumming, and other commonly used for the purification of microbial oils. The crude microbial oil may also be subjected to chemical reactions to produce compound derived from fatty acids, such as esters and salts of DHA or other fatty acids.

As already discussed in relation to the method for reducing the viscosity of a fermentation broth according to the invention, the microbial oil may comprise 30 to 90 wt. % of polyunsatured fatty acids (PUFAs) (calculated on the total weight of fatty acids in the total lipid content, e.g. as determined by FAME analysis), preferably 40 to 85 wt. %, more preferably 50 to 80 wt. %, more preferably 55 to 75 wt. %, optionally of PUFAs comprising three or more double bonds. The microbial oil may comprise 10 to 80 wt. % of DHA (calculated on the total weight of fatty acids in the total lipid content, e.g. as determined by FAME analysis), preferably 20 to 75 wt. %, more preferably 30 to 70 wt. %, more preferably 50 to 65 wt. %. The microbial oil may also comprise 5.0 to 25 wt. % of docosapentaenoic acid (DPA) (based on the total weight of fatty acids in the total lipid content, e.g. as determined by FAME analysis), preferably 10 to 24 wt. %, more preferably 12 to 22 wt. %, still more preferably 14 to 20 wt. %. The microbial oil may also comprise 0.1 to 5.0 wt. % of eicosapentaenoic acid (EPA) (based on the total weight of fatty acids in the total lipid content, e.g. as determined by FAME analysis), preferably 0.2 to 4.0 wt. %, more preferably 0.3 to 3.0 wt. %. The microbial oil may, for example, comprise DHA (in the amounts as defined above) and EPA (in the amounts as defined above). The microbial oil may comprise DHA, EPA, and DPA (all in the amounts as defined above).

### Dried Biomass

Also disclosed herein is a dried biomass comprising microbial cells, 5 wt. % of water or less (calculated on the total weight of the dried biomass), and a total amount of from 0.1 to 20 wt. % of at least one compound selected from the group consisting of ascorbic acid, citric acid, lactic acid, formic acid, amino acids, and soluble salts of said acids (calculated on the dry matter content of the dried biomass), wherein the total lipid content of the dry matter content of the biomass is at least 40 wt. %. The amounts of water and the at least one compound are based on the total weight of the dried biomass.

Preferences set out above for the fermentation broth and the microbial oil also apply to the dried biomass, in particular with regard to the microbial cells and the microbial oil. And vice versa, any preferences discussed below in relation to the dried biomass apply to the dried biomass obtained according to any of the process sequences (b), (c), (e), and (f) discussed above.

The dried biomass comprises the at least one compound in an total amount of from 0.1 to 20 wt. %, preferably from 0.5 to 15 wt. %, more preferably from 1.0 to 5.0 wt. % (calculated on the total weight of the dried biomass). The dried biomass may comprise citric acid in an amount of from 0.1 to 20 wt. %, preferably from 0.5 to 15 wt. %, more preferably from 1.0 to 5.0 wt. % (calculated on the total weight of the dried biomass). The dried biomass may comprise lactic acid in an amount of from 0.1 to 20 wt. %, preferably from 0.5 to 15 wt. %, more preferably from 1.0 to 5.0 wt. % (calculated on the total weight of the dried biomass). The dried biomass may comprise formic acid in an amount of from 0.1 to 20 wt. %, preferably from 0.5 to 15 wt. %, more preferably from 1.0 to 5.0 wt. % (calculated on the dry matter content of the dried biomass). The dried biomass may comprise ascorbic acid in an amount of from 0.1 to 20 wt. %, preferably from 0.5 to 15 wt. %, more preferably from 1.0 to 5.0 wt. % (calculated on the dry matter content of the dried biomass).

The water content of the dried biomass is 5 wt. % or less, e.g. 0.01 to 5 wt. %, (as determined gravimetrically using a moisture analyzer). The water content of the dried biomass is preferably at most 4.0 wt. %, more preferably at most 3.0 wt. %, still more preferably at most 2.0 wt. %, still more preferably at most 1.0 wt. %. The water content of the dried biomass is optionally at least 0.1 wt. %.

The products obtained by the method for processing the fermentation broth according to the invention (e.g., the microbial oil, the dried biomass, and the dispersion comprising lysed microbial cells in oil) can be incorporated in an animal feed, such as a fish feed (preferably a salmon or a shrimp feed). Methods for incorporating biomass into animal feeds are described in, for example, EP 3200604, EP 3200605, and EP 3200605. Methods for incorporating dispersions of lysed microbial cells in oil into feeds are described in, for example, WO 2018005856 and WO 2020141206.

A use of the fermentation broth with a low viscosity (obtained) according to the invention in the preparation of dried biomass, a separated (e.g., extracted) microbial oil, a feed additive, or an animal feed is, thus, also disclosed herein. A use of the dried biomass according to the invention in the preparation of an extracted microbial oil, a feed additive, or an animal feed is also disclosed herein. A use of a compound selected from the group consisting of ascorbic acid, citric acid, lactic acid, formic acid, amino acids, and soluble salts of said acids as a viscosity reducing additive in a fermentation broth comprising microbial cells is also disclosed herein.

When amounts, concentrations, dimensions and other parameters are expressed in the form of a range, a preferable range, an upper limit value, a lower limit value or preferable upper and lower limit values, it should be understood that any ranges obtainable by combining any upper limit or preferable value with any lower limit or preferable value are also specifically disclosed, irrespective of whether the obtained ranges are clearly mentioned in the context. In addition, it should be understood that all percentages mentioned herein are weight percentages, unless specified otherwise.

All documents mentioned herein are incorporated by reference in their entirety or, alternatively, to provide the disclosure for which there were specifically relied upon. As will be evident to the skilled person, different embodiments of the present invention can be combined unless they are mutually exclusive.

### EXAMPLES

The following examples will illustrate the practice of the invention in some preferred embodiments and are not intended to be limiting. Other embodiments within the scope of the invention will be apparent to the skilled person.

### Example 1: Preparing a Fermentation Broth

A Schizochytrium limacinum strain was cultured at about 28° C. in a fermenter with an aeration rate of about 1.0 vvm. Sterilized VHP sucrose syrup (70% w/w) was used as a carbon feedstock, and fed to the culture until the dry matter content was at least 160 g/kg of the fermentation and the lipid content of the dry matter content was 55-70%. The pH was maintained around 5 automatically by the addition of ammonium hydroxide, acid and/or base. Dissolved oxygen (DO) level was maintained at ≥20% of air saturation by automatic control of agitation, pressure and/or oxygen enrichment as needed.

### Example 2: Measuring Certain Parameters of the Fermentation Broth of Example 1

The amount of free microbial oil in a sample is determined gravimetrically, as follows. A sample of fermentation broth is contacted with hot isopropyl alcohol (at 70° C.) at a mass ratio 7.2:1 (hot IPA to broth), mixed and centrifuged at 12,000 rpm for 5 minutes. A subset of the obtained supernatant is transferred to a new container to which water and hexane are added in a 4:3:1 ratio supernatant/hexane/water. The sample is mixed by inversion for 10 seconds and then centrifuged for 1 minute at 12,000 rpm. The hexane layer is carefully pipetted out to a smooth aluminum tray and evaporated in a fumehood. The obtained amount of oil is weighed and compared to the weight of the starting sample. The fermentation broth obtained in Example 1 had a free oil content of 0.5 wt. % of the total fermentation broth, measured with the said method.

The total lipid of a sample (e.g., dried biomass, a liquid suspension, extracted oil, or dried broth) is determined as follows by performing a FAME analysis. Samples were analyzed for fatty acid composition by converting them to FAMEs using direct transesterification. Samples (10-20 mg) were weighed directly into 16×100 mm test tubes with PTFE-lined screw caps, followed by 200 μL of a 20 mg/mL solution of C19:0 internal standard (NuChek Prep, Inc, Elysian, MN) and 2 mL of 5% sulfuric acid in methanol containing 0.05% BHT. The tubes were capped and placed in a dry bath maintained at 75° C. for 3.5 h and vortexed and sonicated in a sonicator bath heated to 75° C. twice intermittently during the course of the transesterification. Sample tubes were removed from the dry block, and once cooled to room temperature, 2 mL of 10% potassium phosphate tribasic and 2 mL of heptane were added to the tube. The sample tubes were agitated vigorously with inversion and then centrifuged at 1600 rpm for 2 min to provide two distinct layers. A suitable portion of the upper layer was transferred to a sample vial or vial insert which contained sodium sulfate (anhydrous) lining the bottom, for analysis by GC-FID. FAMEs were quantified using empirical relative response factors (ERRF) for each FAME identified relative to the C19:0 internal standard, following the acceptable ERRF criteria guidelines outlined in AOCS method Ce 1i-07. The fermentation broth in Example 1 had a total lipid content of 55-70 wt. %, as already mentioned in Example 1.

### Example 3: Reducing Viscosity with Salts of Citric Acid

Antioxidants were added to the fermentation broth at pH 5 under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of salt (g/kg total broth) was added to the broth under mixing to the broth and kept stirred for 15 minutes after which pH was measured. A subset of the sample was used to determine viscosity at 25° C. using a Malvern Kinexus Pro+ rheometer. The viscosities were compared and relative viscosity reduction was calculated comparing to the control broth sample.

| | | | Relative viscosity |
|---|---|---|---|
| Example | Salt | pH of the broth | reduction |
| 2A | Na-citrate dihydrate | 5.4 | 42% |
| | (20 g/kg) | | |
| 2B | Mg-citrate dihydrate | 4.9 | 46% |
| | (20 g/kg) | | |

The viscosity of the fermentation broth after addition of the salt was 1.0 Pa·s or less measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

### Example 3: Reducing Viscosity with Calcium Formate

Two antioxidants were added to the fermentation broth at pH 5 under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of salt (g/kg total broth) was added to the broth under mixing to the broth and kept stirred for 15 minutes after which pH was measured. A subset of the sample was used to determine viscosity at 25° C. using a Malvern Kinexus Pro+ rheometer. The viscosities were compared and relative viscosity reduction was calculated comparing to the control broth sample.

| | | | Relative viscosity |
|---|---|---|---|
| Example | Salt | pH of the broth | reduction |
| 3A | Ca-formate anhydrous | 5.1 | 28% |
| | (20 g/kg) | | |

The viscosity of the fermentation broth after addition of the salt was 1.0 Pa·s or less measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

### Example 4: Reducing Viscosity with Ascorbic Acid

Antioxidants were added to the fermentation broth at pH 5 to the solids amount under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of ascorbic acid (g/kg total broth) was added to the broth under mixing to broth and kept stirred for 15 minutes after which pH was measured. A first sample was taken after addition of in total 10 g/kg ascorbic acid and its viscosity was measured at 25° C. using a Malvern Kinexus Pro+ rheometer (Example 4A). A second sample was taken after addition of in total 15 g/kg ascorbic acid and its viscosity was also measured using the same method (Example 4B). A subset of the sample with in total 15 g/kg ascorbic acid was taken and pH was reduced to 4.2 using concentrated sulfuric acid. Viscosity of that sample was determined at 25° C. using a Malvern Kinexus Pro+ rheometer (Example 4C). The viscosities were compared and relative viscosity reduction was calculated comparing to the control broth sample.

| | | | Relative viscosity |
|---|---|---|---|
| Example | Salt | pH of the broth | reduction |
| 4A | Ascorbic acid (10 g/kg) | 4.6 | 20% |
| 4B | Ascorbic acid (15 g/kg) | 4.5 | 33% |
| 4C | Ascorbic acid (15 g/kg) | 4.5 (then lowered | 41% |
| | | with sulfuric acid | |
| | | to 4.2) | |

The viscosity of the fermentation broth after addition of the salt was 1.0 Pa·s or less measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

This experiment shows that organic acids can reduce viscosity and that further reduction can be achieved if the pH is lowered.

### Example 5: Reducing Viscosity with D-Lactic Acid

Antioxidants were added to the fermentation broth at pH 5 at to the solids amount under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of lactic acid (g/kg total broth) was added to the broth under mixing and kept stirred for 15 minutes after which pH was measured. A subset of the sample was used to determine viscosity at 25° C. using a Malvern Kinexus Pro+ rheometer (Example 5A). A subset of the sample with lactic acid was taken and pH was increased to 5.0 using 25% sodium hydroxide base. Viscosity of that sample was determined at 25° C. using a Malvern Kinexus Pro+ rheometer (Example 5B). The viscosities were compared and relative viscosity reduction was calculated comparing to the control broth sample.

| | | | Relative viscosity |
|---|---|---|---|
| Example | Salt | pH of the broth | reduction |
| 5A | D-lactic acid (30 g/kg) | 3.2 | 56% |
| 5B | D-lactic acid (30 g/kg) | 3.2, then increased | 61% |
| | | with base to 5.00 | |

The viscosity of the fermentation broth after addition of the salt was 1.0 Pa·s or less measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

This experiment shows that organic acids reduce viscosity and that reduction is not only pH dependent.

### Example 6: Reducing Viscosity with a Combination of Magnesium Citrate and Calcium Lactate

Antioxidants were added to the fermentation broth at pH 5 under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of the below specified amounts of citrate and dilactate (g/kg total broth) were added to the broth under mixing to the broth and kept stirred for 15 minutes after which pH was measured. A subset of the sample was used to determine viscosity at 25° C. using a Malvern Kinexus Pro+ rheometer (Example 6A). Another subset of the sample was taken and pH was decreased to pH=4 using concentrated sulfuric acid (Example 6B). Yet another subset of the sample was taken and its pH decreased to pH=3 using concentrated sulfuric acid and subsequently increased to pH 4 with 25% sodium hydroxide base (Example 6C). Viscosity of the samples was determined at 25° C. using a Malvern Kinexus Pro+ rheometer. The viscosities were compared and relative viscosity reduction was calculated comparing to the control broth sample.

| | | | Relative viscosity |
|---|---|---|---|
| Example | Salt | pH of the broth | reduction |
| 6A | Mg-citrate dihydrate | 5 | 39% |
| | (5 g/kg) | | |
| | Ca-dilactate | | |
| | pentahydrate (5 g/kg) | | |
| 6B | Mg-citrate dihydrate | 5, then reduced | 51% |
| | (5 g/kg) | with acid to pH 4 | |
| | Ca-dilactate | | |
| | pentahydrate (5 g/kg) | | |
| 6C | Mg-citrate dihydrate | 5, then reduced to | 58% |
| | (5 g/kg) | pH 3 using an acid | |
| | Ca-dilactate | and increased with | |
| | pentahydrate (5 g/kg) | based to pH 4 | |

The viscosity of the fermentation broth after addition of the salt was 1.0 Pa·s or less, measured at a shear rate of 9.8-10.0 s⁻¹ and 25° C., as determined according to a shear rate logarithmic ramp up and down isothermal sweep (shear rate range 0.1-100 s⁻¹) using a Malvern Kinexus Pro+ rheometer with a cup and bob system.

This shows that viscosity reduction can be achieved combining two salts of carboxylic acids. It is noteworthy that, when a combination of two salts of carboxylic acids is used, the total amount of salt added can be reduced. This is, of course, economical and environmentally friendly.

It can be observed that addition of salts can reduce viscosity and that pH reduction amplifies this effect. However, a “pH swing” can further contribute to viscosity reduction effects, comparing examples 6B and 6C.

### Example 7 (not According to the Invention): Increasing Viscosity with Salts of Acids

Antioxidants were added to the fermentation broth at pH 5 under mixing at room temperature and kept stirred for 30 minutes. The viscosity was determined on a subset of the broth sample, used as control, at 25° C. using a Malvern Kinexus Pro+ rheometer. Sample was measured using a cup and bob sample with 4 ml sample volume in a shear rate range of 0.1-100 s⁻¹ and the viscosity value at 9.8 s⁻¹ was taken as representative viscosity. The below listed concentration of salt (g/kg total broth) was added to the broth under mixing to the broth and kept stirred for 15 minutes after which pH was measured. A subset of the sample was used to determine viscosity at 25° C. using a Malvern Kinexus Pro+ rheometer. The viscosities were compared and relative viscosity reduction was calculated comparing to the control broth sample.

| | | | Change in relative |
|---|---|---|---|
| Example | Salt | pH of the broth | viscosity |
| 7A | Na₂-fumarate (10 g/kg) | 5.2 | Increased viscosity |
| 7B | Na₂-malate (10 g/kg) | 5.3 | Increased viscosity |
| 7C | Mg-acetate (10 g/kg) | 5 | Increased viscosity |
| 7D | Na₂-oxalate (10 g/kg) | 5 | Increased viscosity |

This shows that not all organic acids, when added to a fermentation broth as used in the method according to the invention, decrease the viscosity of the fermentation broth.

Also disclosed herein are the following clauses:

Clause 1. Method for reducing the viscosity of a fermentation broth, wherein the method comprises the step of
mixing at least one compound selected from the group consisting of ascorbic acid, citric acid, lactic acid, formic acid, amino acids, and soluble salts of said acids into a fermentation broth comprising microbial cells, the fermentation broth having free microbial oil in an amount of 5.0 wt. % or less, calculated on the total weight of the fermentation broth, wherein the total lipid content of the dry matter content of the fermentation broth is at least 40 wt. %.

Clause 2. The method according to clause 1, further comprising the step(s) of
decreasing or increasing the pH of the fermentation broth; and
re-adjusting the pH of the fermentation broth in the opposite direction (i.e., increasing or decreasing the pH, respectively) by addition of a base or an acid.

Clause 3. The method according to clause 1 or 2, wherein the method further comprises adding a further acid, preferably a further acid selected from the group consisting of sulfuric acid, phosphoric acid, and nitric acid.

Clause 4. The method according to any one of clauses 1 to 3, wherein the soluble salt is a (di) sodium salt, a magnesium salt, a calcium salt, a (di) potassium salt, a (di) lithium salt, or an ammonium salt.

Clause 5. The method according to any one of clauses 1 to 4, wherein the at least one compound is mixed into the fermentation broth in a total amount of from 0.05 wt. % to 10 wt. % (calculated on the dry matter content of the fermentation broth), preferably 0.10 wt. % to 7.0 wt. %, more preferably 0.15 wt. % to 5.0 wt. %.

Clause 6. The method according to any one of clauses 1 to 5, wherein the at least one compound is selected from the group consisting of ascorbic acid, citric acid, lactic acid, formic acid, and soluble salts of said acids, more preferably from the group consisting of ascorbic acid, citric acid, lactic acid, and soluble salts of said acids, more preferably ascorbic acid and soluble salts thereof.

Clause 7. The method according to any one of clauses 1 to 6, wherein the viscosity of the fermentation broth after the at least one compound has been mixed thereinto is at least 10% lower than the viscosity of the fermentation broth before the compound was mixed thereinto, preferably at least 25% lower, more preferably at least 40% lower.

Clause 8. The method according to any one of clauses 1 to 7, wherein the microbial cells are from the family Thraustochytriaceae, preferably from the genus Schizochytrium, Thraustochytrium, Aurantiochytrium, or Ulkenia, or from the family Crypthecodiniaceae, preferably from the genus Crypthecodinium.

Clause 9. The method according to any one of clauses 1 to 8, wherein the microbial cells comprise a microbial oil that comprises:
30 to 90 wt. % of polyunsatured fatty acids (PUFAs) (calculated on the total weight of fatty acids in the total lipid content), preferably 40 to 85 wt. %, more preferably 50 to 80 wt. %, more preferably 55 to 75 wt. %, optionally of PUFAs comprising three or more double bonds;
40 to 80 wt. % of DHA docosahexaenoic acid (DHA), preferably 45 to 75 wt. % DHA, more preferably 50 to 65 wt. % DHA (calculated on the total weight of fatty acids in the total lipid content); and/or
0.1 to 5.0 wt. % of eicosapentaenoic acid (EPA), preferably 0.2 to 4.0 wt. %, more preferably 0.3 to 3.0 wt. %, (calculated on the total weight of fatty acids in the total lipid content).

Clause 10. The method according to any one of clauses 1 to 9, wherein the amount of free microbial oil in the fermentation broth is 4.0 wt. % or less, preferably 3.0 wt. % or less, more preferably 2.0 wt. % or less, more preferably 1.0 wt. % or less, more preferably 0.9 wt. % or less, preferably 0.8 wt. % or less, still more preferably 0.6 wt. % or less, still more preferably 0.6 wt. % or less, still more preferably 0.5 wt. % or less.

Clause 11. The method according to any one of clauses 1 to 10, wherein the dry matter content of the fermentation broth is at least 10 wt. %, preferably at least 11 wt. %, more preferably at least 12 wt. %, still more preferably at least 13 wt. %, still more preferably at least 14 wt. %, still more preferably at least 15 wt. % (calculated on the total weight of the fermentation broth).

Clause 12. The method according to any one of clauses 1 to 11, wherein, 15 minutes after addition of the at least one compound, the amount of free microbial oil in the fermentation broth is at most 5.0 wt. %.

Clause 13. Fermentation broth comprising microbial cells, at least one compound selected from the group consisting of ascorbic acid, citric acid, lactic acid, formic acid, amino acids, and soluble salts of said acids, and 5.0 wt. % or less of free microbial oil (calculated on the total weight of the fermentation broth), wherein the total lipid content of the dry matter content of the fermentation broth is at least 40 wt. %.

Clause 14. The fermentation broth according to clause 13, wherein the fermentation broth has a viscosity of 1.0 Pa·s or less, preferably less than 0.5 Pas, more preferably 0.2 Pa·s, even more preferably 0.1 Pa·s, measured at a shear rate of 9.8-10.0 s 1, 25° C., and according to a shear rate logarithmic ramp up and down isothermal sweep (shear rate range 0.1-100 s⁻¹) using a Malvern Kinexus Pro+ rheometer with a cup and bob system.

Clause 15. The fermentation broth according to clauses 13 or 14, wherein the fermentation broth comprises a total amount of from 0.05 wt. % to 10 wt. % of the at least one compound (calculated on the dry matter content of the fermentation broth).

Clause 16. The fermentation broth according to any one of clauses 13 to 15, wherein the microbial cells are from the family Thraustochytriaceae, preferably from the genus Schizochytrium, Thraustochytrium, Aurantiochytrium, or Ulkenia, or from the family Crypthecodiniaceae, preferably from the genus Crypthecodinium.

Clause 17. The fermentation broth according to any one of clauses 13 to 16, wherein the microbial cells comprise a microbial oil comprising 40 to 80 wt. % of DHA docosahexaenoic acid (DHA), preferably 45 to 75 wt. % DHA, more preferably 50 to 70 wt. % DHA (calculated on the total weight of fatty acids in the total lipid content).

Clause 18. The fermentation broth according to any one of clauses 13 to 17, wherein the dry matter content of the fermentation broth is at least 10 wt. %, preferably at least 11 wt. %, more preferably at least 12 wt. %, still more preferably at least 13 wt. %, still more preferably at least 14 wt. %, still more preferably at least 15 wt. % (calculated on the total weight of the fermentation broth).

Clause 19. The fermentation broth according to any one of clauses 13 to 18, wherein, 15 minutes after addition of the at least one compound, the amount of free microbial oil in the fermentation broth is at most 5.0 wt. %.

Clause 20. Method for processing a fermentation broth obtained according to the method of any one of clauses 1 to 12 or a fermentation broth according to any one of clauses 13 to 19 comprising one of the process sequences
(a) concentrating the fermentation broth (e.g. by evaporation (of water), filtration, or centrifugation) to obtain a concentrated fermentation broth;
(b) concentrating the fermentation broth (e.g. by evaporation (of water), filtration, or centrifugation) to obtain a concentrated fermentation broth, and, thereafter, drying the concentrated fermentation broth to obtained a dried biomass;
(c) concentrating the fermentation broth (e.g. by evaporation (of water), filtration, or centrifugation) to obtain a concentrated fermentation broth, drying the concentrated fermentation broth to obtained a dried biomass, and lysing the dried biomass (optionally in the presence of oil) to obtain a dispersion comprising lysed microbial cells in oil;
(d) concentrating the fermentation broth (e.g. by evaporation (of water), filtration, or centrifugation) to obtain a concentrated fermentation broth, lysing microbial cells in the concentrated fermentation broth to obtain a concentrated fermentation broth comprising lysed microbial cells, and separating microbial oil from the concentrated fermentation broth comprising lysed microbial cells in one or more steps, optionally without performing a demulsification step (e.g., adding an additional demulsification agent) before separating the microbial oil;
(e) drying the fermentation broth (without first concentrating the fermentation broth) to obtain a dried biomass;
(f) drying the fermentation broth (without first concentrating the fermentation broth) to obtain a dried biomass and, thereafter, lysing the dried biomass (optionally in the presence of oil) to produce a dispersion comprising lysed microbial cells in oil;
(g) lysing microbial cells in the fermentation broth to obtain a fermentation broth comprising lysed cells;
(h) lysing microbial cells in the fermentation broth to obtain a fermentation broth comprising lysed cells and separating microbial oil from the fermentation broth comprising lysed cells in one or more steps, optionally wherein a demulsification step is performed prior to separating microbial oil from the fermentation broth comprising lysed cells; or
(i) lysing microbial cells in the fermentation broth to obtain a fermentation broth comprising lysed cells, concentrating the fermentation broth comprising lysed cells (e.g., by evaporation (of water) or centrifugation), and separating microbial oil from the fermentation broth comprising lysed cells in one or more steps, optionally wherein a demulsification step is performed prior to separating microbial oil from the fermentation broth comprising lysed cells.

Clause 21. The method of clause 20, wherein the method further comprises the step of incorporating the dried biomass, the lysed biomass, the dispersion of lysed microbial cells in oil, or the microbial oil in an animal feed, preferably a fish feed, more preferably a salmon feed.

Clause 22. Dried biomass comprising microbial cells, 5 wt. % of water or less (calculated on the total weight of the dried biomass), and a total amount of from 0.1 to 20 wt. % of at least one compound selected from the group consisting of ascorbic acid, citric acid, lactic acid, formic acid, amino acids, and soluble salts of said acids (calculated on the total weight of the dried biomass), wherein the total lipid content of the dry matter content of the biomass is at least 40 wt. %.

Clause 23. Use of a fermentation broth obtained according to the method of any one of clauses 1 to 12 or a fermentation broth according to any one of clauses 13 to 19 in the preparation of dried biomass, an extracted microbial oil, a feed additive, or an animal feed; or use of the dried biomass according to clause 22 in the preparation of an extracted microbial oil, a feed additive, or an animal feed.

Clause 24. Use of ascorbic acid, or a soluble salt thereof, as a viscosity reducing additive in a fermentation broth comprising microbial cells.

Clause 25. The use according to clause 24, wherein the fermentation broth has a dry matter content of at least 10 wt. % (calculated on the total weight of the fermentation broth), preferably at least 11 wt. %, more preferably at least 12 wt. %, still more preferably at least 13 wt. %, still more preferably at least 14 wt. %, still more preferably at least 15 wt. %.

Clause 26. The use according to clause 24 or 25, wherein ascorbic acid is used in an amount of 0.05-10 wt. % (calculated on the total weight of the fermentation broth), preferably 0.1-7.0 wt. %, more preferably 0.15-5.0 wt. %.

Clause 27. The use according to any one of clauses 24 to 26, wherein the microbial cells are from the family Thraustochytriaceae or Crypthecodiniaceae.

Clause 28. The use according to clause 27, wherein the microbial cells are from the genus Schizochytrium, Thraustochytrium, Aurantiochytrium, or Ulkenia, or from the genus Crypthecodinium.

Clause 29. The use according to any one of clauses 24 to 28, wherein the fermentation broth has 5.0 wt. % or less of free microbial oil (calculated on the total weight of the fermentation broth) and wherein the total lipid content of the dry matter content of the fermentation broth is at least 40 wt. %.

Clause 30. The use according to any one of clauses 24 to 29, wherein the soluble salt is a sodium salt, a magnesium salt, a calcium salt, a potassium salt, a lithium salt, or an ammonium salt.

Clause 31. The use according to any one of clauses 24 to 30, wherein the ascorbic acid or soluble salt thereof is used in combination with a second acid, preferably wherein the second acid is selected from the group consisting of citric acid, lactic acid, formic acid, amino acids, and soluble salts or from the group consisting of sulfuric acid, phosphoric acid, and nitric acid.

Clause 32. Use of citric acid, lactic acid, formic acid, amino acids, or a soluble salt thereof, as a viscosity reducing additive in a fermentation broth comprising microbial cells.

Clause 33. The use according to clause 32, wherein the fermentation broth has a dry matter content of at least 10 wt. % (calculated on the total weight of the fermentation broth), preferably at least 11 wt. %, more preferably at least 12 wt. %, still more preferably at least 13 wt. %, still more preferably at least 14 wt. %, still more preferably at least 15 wt. %.

Clause 34. The use according to clause 32 or 33, wherein ascorbic acid is used in an amount of 0.05-10 wt. % (calculated on the total weight of the fermentation broth), preferably 0.1-7.0 wt. %, more preferably 0.15-5.0 wt. %.

Clause 35. The use according to any one of clauses 32 to 34, wherein the microbial cells are from the family Thraustochytriaceae or Crypthecodiniaceae.

Clause 36. The use according to clause 35, wherein the microbial cells are from the genus Schizochytrium, Thraustochytrium, Aurantiochytrium, or Ulkenia, or from the genus Crypthecodinium.

Clause 37. The use according to any one of clauses 32 to 36, wherein the fermentation broth has 5.0 wt. % or less of free microbial oil (calculated on the total weight of the fermentation broth) and wherein the total lipid content of the dry matter content of the fermentation broth is at least 40 wt. %.

Clause 38. The use according to any one of clauses 32 to 37, wherein the soluble salt is a (di) sodium salt, a magnesium salt, a calcium salt, a (di) potassium salt, a (di) lithium salt, or an ammonium salt.

1. A method for reducing the viscosity of a fermentation broth, the method comprising (a) mixing at least one compound selected from the group consisting of ascorbic acid, citric acid, lactic acid, formic acid, amino acids, and soluble salts of the acids with a fermentation broth comprising microbial cells, the fermentation broth having free microbial oil in an amount of 5.0 wt. % or less, calculated on the total weight of the fermentation broth, wherein the total lipid content of the dry matter content of the fermentation broth is at least 40 wt. %.

2. The method according to claim 1, further comprising:
(b) decreasing or increasing the pH of the fermentation broth; and
(c) re-adjusting the pH of the fermentation broth by adding a base or an acid.

3. The method according to claim 1, further comprising adding a further acid selected from the group consisting of sulfuric acid, phosphoric acid, and nitric acid.

4. The method according to claim 1, wherein the soluble salt is a (di) sodium salt, a magnesium salt, a calcium salt, a (di) potassium salt, a (di) lithium salt, or an ammonium salt.

5. The method according to claim 1, wherein the at least one compound is mixed into the fermentation broth in a total amount of from 0.05 wt. % to 10 wt. % (calculated on the total weight of the fermentation broth).

6. The method according to claim 5, wherein the at least one compound is mixed into the fermentation broth in a total amount of from 0.15 wt. % to 50 wt. % (calculated on the total weight of the fermentation broth).

7. The method according to claim 1, wherein the at least one compound is selected from the group consisting of ascorbic acid, citric acid, lactic acid, and soluble salts of the acids.

8. The method according to claim 1, wherein the fermentation broth after the at least one compound has been mixed thereinto has a viscosity at least 10% lower than the viscosity of the fermentation broth before the compound was mixed thereinto.

9. The method according to claim 8, wherein the fermentation broth after the at least one compound has been mixed thereinto has a viscosity at least 40% lower than the viscosity of the fermentation broth before the compound was mixed thereinto.

10. The method according to claim 1, wherein the microbial cells are from the family Thraustochytriaceae or Crypthecodiniaceae,

11. The method according to claim 10, wherein the microbial cells are from the genus Schizochytrium, Thraustochytrium, Aurantiochytrium, Ulkenia, or Crypthecodinium.

12. The method according to claim 1, wherein the microbial cells comprise a microbial oil that comprises:
30 to 90 wt. % of polyunsatured fatty acids (PUFAs) (calculated on the total weight of fatty acids in the total lipid content), optionally of PUFAs comprising three or more double bonds;
40 to 80 wt. % of DHA docosahexaenoic acid (DHA); and/or
0.1 to 5.0 wt. % of eicosapentaenoic acid (EPA), (calculated on the total weight of fatty acids in the total lipid content).

13. The method according to claim 12, wherein the microbial cells comprise a microbial oil that comprises:
55 to 75 wt. % of polyunsatured fatty acids (PUFAs) (calculated on the total weight of fatty acids in the total lipid content), optionally of PUFAs comprising three or more double bonds;
50 to 65 wt. % of DHA docosahexaenoic acid (DHA); and/or
0.3 to 3.0 wt. % of eicosapentaenoic acid (EPA) (calculated on the total weight of fatty acids in the total lipid content).

14. The method according to claim 1, wherein the amount of free microbial oil in the fermentation broth is 4.0 wt. % or less; and/or wherein, 15 minutes after addition of the at least one compound, the amount of free microbial oil in the fermentation broth is at most 5.0 wt. %.

15. The method according to claim 1, wherein the amount of free microbial oil in the fermentation broth is 1.0 wt. % or less.

16. The method according to claim 1, wherein the dry matter content of the fermentation broth is at least 10 wt. % (calculated on the total weight of the fermentation broth).

17. A fermentation broth comprising:
(a) microbial cells,
(b) at least one compound selected from the group consisting of ascorbic acid, citric acid, lactic acid, formic acid, amino acids, and soluble salts of the acids, and
(c) 5.0 wt. % or less of free microbial oil (calculated on the total weight of the fermentation broth),
wherein the total lipid content of the dry matter content of the fermentation broth is at least 40 wt. %.

18. A method for processing a fermentation broth according to claim 17, comprising one or more of:
(a) concentrating the fermentation broth to obtain a concentrated fermentation broth;
(b) concentrating the fermentation broth to obtain a concentrated fermentation broth, and, thereafter, drying the concentrated fermentation broth to obtained a dried biomass;
(c) concentrating the fermentation broth to obtain a concentrated fermentation broth, drying the concentrated fermentation broth to obtained a dried biomass, and lysing the dried biomass, optionally in the presence of oil, to obtain a dispersion comprising lysed microbial cells in oil;
(d) concentrating the fermentation broth to obtain a concentrated fermentation broth, lysing microbial cells in the concentrated fermentation broth to obtain a concentrated fermentation broth comprising lysed microbial cells, and separating microbial oil from the concentrated fermentation broth comprising lysed microbial cells in one or more steps, optionally without performing a demulsification step before separating the microbial oil;
(e) drying the fermentation broth, without first concentrating the fermentation broth, to obtain a dried biomass;
(f) drying the fermentation broth, without first concentrating the fermentation broth, to obtain a dried biomass and, thereafter, lysing the dried biomass, optionally in the presence of oil, to produce a dispersion comprising lysed microbial cells in oil;
(g) lysing microbial cells in the fermentation broth to obtain a fermentation broth comprising lysed cells;
(h) lysing microbial cells in the fermentation broth to obtain a fermentation broth comprising lysed cells and separating microbial oil from the fermentation broth comprising lysed cells in one or more steps, optionally wherein a demulsification step is performed prior to separating microbial oil from the fermentation broth comprising lysed cells; or
(i) lysing microbial cells in the fermentation broth to obtain a fermentation broth comprising lysed cells, concentrating the fermentation broth comprising lysed cells, and separating microbial oil from the fermentation broth comprising lysed cells in one or more steps, optionally wherein a demulsification step is performed prior to separating microbial oil from the fermentation broth comprising lysed cells.

19. The method for processing a fermentation broth according to claim 18, wherein the concentrating comprises evaporation, filtration, or centrifugation.

20. A dried biomass comprising microbial cells, 5 wt. % of water or less, calculated on the total weight of the dried biomass, and a total amount of from 0.1 to 20 wt. % of at least one compound selected from the group consisting of ascorbic acid, citric acid, lactic acid, formic acid, amino acids, and soluble salts of said acids, calculated on the total weight of the dried biomass, wherein the total lipid content of the dry matter content of the biomass is at least 40 wt. %.
